(19) United States
(12) Patent Application Publication
(10) Pub. No.: US 2008/0201103 A1
(43) Pub. Date: Aug. 21, 2008

(54) Robust Detection of Variability in Multiple Sets of Data

(75) Inventors: Chengyong Yang, Foster City, CA (US); David C. Woo, Foster City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 12/021,032

(22) Filed: Jan. 28, 2008

Related U.S. Application Data
(60) Provisional application No. 60/898,303, filed on Jan. 30, 2007.

(51) Int. Cl. G06F 17/18; G01N 33/48; G01D 18/00

(52) U.S. Cl. 702/179; 702/19; 702/85

(57) ABSTRACT

The present teachings comprise systems and methods for calibrating the background or baseline signal in a PCR or other reaction. The background signal derived from detected emissions of sample wells can be subjected to a normalized statistical metric, and be compared to a threshold or other standard to discard outlier cycles or other extraneous data. According to various embodiments, a relative standard deviation (relativeSTD) for the background component can be generated by dividing the standard deviation by the median of differences across all wells, where the difference is defined as the difference between maximum and minimum pixel values of a well. The relativeSTD as a metric is not sensitive to machine-dependent variations in absolute signal output that can be caused by different gain settings, different LED draw currents, different optical paths, or other instrumental variations. More accurate background characterization can be achieved.

### RELATED APPLICATION

This application claims priority to U.S. Provisional Patent Application No. 60/898,303, filed Jan. 30, 2007, entitled “Background Calibration with Robust Detection of Contamination and Background Variability,” which is incorporated by reference herein in its entirety.

### BACKGROUND

The detection and calibration of the background fluorescent signal in spectral instruments is important for the performance of the instrument, and for the integrity of detection and analytic processes carried out on the instrument. For example, calibrating the background fluorescent signal or noise floor is important for dye calibration in fluorescent detection systems. Calibrating the fluorescent background is also important in actual sample runs, since those runs can involve subtraction of the background baseline to remove effects due to non-amplified products or noise floor. Background calibration can furthermore be important to check for background uniformity, and to consistently detect outlier wells. Background calibration can, for example, be used to estimate the emission noise floor registered by the optical detection system of a polymerase chain reaction (PCR) amplification instrument, or other instrument or device.

When applied to PCR instruments, the background noise floor can for instance be subtracted from the detected amplification curve or profile of one or more fluorescently labeled amplification products, such as labeled genetic or other sample material. PCR instruments can generally be configured to identify a baseline region, exponential amplification region, and plateau region of emission data captured during the PCR amplification cycle. Detection of signal peaks in the amplification profile or other signal detection output can also be performed, to generate a peak calibration that calibrates peak intensities and/or positions in sample plate or other support.

Background calibration is generally performed after peak calibration, since the background calibration analysis can require the peak location information. The background calibration run can collect several cycles of data, and collect data ftom several spectral filters or channels. For example, a background calibration run can collect ten cycles of data from three filters, for example three filters configured to pass light of three difference wavelengths generally corresponding to the emission wavelengths of different fluorescent dye labels. In that case, the background run will produce thirty images.

Background calibration to identify a fluorescent or other baseline signal can be performed on a per-well basis. The fluorescent signal from a well can be calculated by summing pixel values captured around the peak intensity position of that well. For each well, the background signal calibration can be calculated by averaging all the detection cycles. Current calibration analysis, however, does not test whether data from all the cycles are acceptable beyond checking whether the background signal is within a specified range. Also, current calibration techniques do not check whether signals from all wells in the same plate are distributed uniformly. It is possible that individual wells or clusters of wells can suffer contamination from particulate debris, extraneous dyes, markers, or inks, cloth, dust, reagent or other spills, or other contaminants. When contamination occurs, emission data from one or more contaminated wells can be shifted or degraded significantly, for instance, to display reduced intensity or distorted spectra. If there is any contamination in one or more wells the block or plate used for calibrating the fluorescent background, current calibration analysis does not detect or report it. Background levels can therefore be erroneously or inaccurately recorded, and then, for instance, used to baseline PCR or other sample runs, potential skewing or corrupting the results.

Current background calibration analysis can further be sensitive to instrument factors such as variations in instrument offset, detection system gain settings, LED power output and current settings, and filter efficiency. These and other background calibration parameters can be different and vary differently across instruments, and must be set in the factory or in the field by field specialists. Techniques to estimate the background distribution, check background uniformity, and consistently detect outlier wells across all units, but that are insensitive to these and other instrument factors, are therefore needed.

### SUMMARY

According to various embodiments of the present teachings, systems and methods are provided wherein detected raw background signal data is received and processed to generate a normalized background calibration profile for PCR and other detection systems. According to various embodiments, the normalized background calibration profile can compensate for corrupt or contaminated wells and process cycles, while remaining insensitive to instrumental variations such as detector gain, illumination source current, filter efficiency, and other factors. In some embodiments, a conventional standard deviation measure can be generated for detected well intensities, entire cycles or plates, or separate filter measurements, and that measure can then be subjected to further conditioning by, for example, dividing by a mean of the well intensity range of difference. According to various embodiments, the relative standard deviation (relativeSTD) measure can be used to characterize raw background data and to compare, analyze, modify, and condition background calibration profiles on a uniform basis. In some embodiments, data points that fail to conform to predetermined thresholds or other standards can be excluded from the analysis, and therefore improve the accuracy of the background calibration itself and PCR or other processes using the background calibration profile.

According to various embodiments, a method of generating a detection signal from a reaction is provided. The method comprises receiving reaction emission data comprising multiple sets of data; and generating a relative standard deviation (relativeSTD) representing a normalized measure of variation in the multiple sets of data, by applying the equation:

$$relativeSTD = \frac{STD}{MedianDiffMinPeak} ,$$

where STD comprises the standard deviation of the emission data, MedianDiffMinPeak comprises the median of differences across the multiple sets of data, and each of the differences comprises the difference between a respective detected maximum value and a respective detected minimum value for each respective set of data of the multiple sets of data. The method can further comprise determining whether to exclude one or more of the multiple sets of data based on the normalized measure; excluding sets of data from the multiple sets of data, having relativeSTD values that exceed a predetermined value, to generate processed emission data; and generating a detection signal based on the processed emission data. In some embodiments, the receiving reaction emission data comprising multiple sets of data can comprise receiving emission data and generating multiple sets of data from the data received. In some embodiments, the emission data can comprise emission data detected from a biological sample. The reaction from which the detection signal is generated can comprise a polymerase chain reaction. In some embodiments, the reaction comprises a reaction with a nucleic acid. In some embodiments, the receiving emission data can comprise receiving emission data from a plurality of sample wells. In some embodiments, the emission data can comprise data detected using a plurality of filters, and generating a relativeSTD can comprise generating a relativeSTD on a per-filter basis.

According to various embodiments, a system for generating a detection signal from a reaction is provided. The system can comprise an input unit configured to receive reaction emission data comprising multiple sets of data; and a processor unit. The processor unit can be configured to generate a relative standard deviation (relativeSTD) representing a normalized measure of variation in the multiple sets of data, by applying the equation:

$$relativeSTD = \frac{STD}{MedianDiffMinPeak} ,$$

where STD comprises the standard deviation of the emission data, MedianDiffMinPeak comprises the median of differences across the multiple sets of data, and each of the differences comprises the difference between a respective detected maximum value and a respective detected minimum value for each respective set of data of the multiple sets of data. The processor unit can also be configured to determine whether to exclude one or more of the multiple sets of the data based on the normalized measure, and to exclude sets of data from the multiple sets of data, having relativeSTD values that exceed a predetermined value, to generate processed emission data. In some embodiments, the processor unit can generate a detection signal based on the processed emission data. In some embodiments, the emission data that the input unit is configured to receive can comprise emission data detected from labeled nucleic acid samples. In some embodiments, the input unit can be configured to receive emission data from a polymerase chain reaction. In some embodiments, the input unit can comprise a plurality of different filters configured to filter the emission data from the reaction.

According to various embodiments, a background calibration profile is provided and can be generated by a method that comprises: receiving reaction emission data comprising multiple sets of data, from an amplification reaction having cycles; and generating a relative standard deviation (relativeSTD) representing a normalized measure of variation in the multiple sets of data, by applying the equation:

$$relativeSTD = \frac{STD}{MedianDiffMinPeak} ,$$

where STD comprises the standard deviation of the emission data, MedianDiffMinPeak comprises the median of differences across the multiple sets of data, and each of the differences comprises the difference between a respective detected maximum value and a respective detected minimum value for each respective set of data of the multiple sets of data. In some embodiments, the method can comprise determining whether to exclude one or more of the multiple sets of data based on the normalized measure, and excluding sets of data from the multiple sets of data, having relativeSTD values that exceed a predetermined value, to generate processed emission data. In some embodiments, the method can comprise generating a background calibration profile based on the processed emission data. The emission data can comprise emission data detected from labeled nucleic acid samples, for example, from fluorescently labeled DNA samples. In some embodiments, the amplification reaction can comprise a polymerase chain reaction. In some embodiments, the receiving emission data can comprise receiving emission data from DNA samples loaded in a plurality of sample wells. In some embodiments, the receiving reaction emission data comprising multiple sets of data can comprise generating multiple sets of data from received emission data.

According to yet other various embodiments, a computer-readable medium is provided that is readable to execute a method of generating a detection signal from a reaction. The method can comprise: receiving reaction emission data comprising multiple sets of data; and generating a relative standard deviation (relativeSTD) representing a normalized measure of variation in the multiple sets of data, by applying the equation:

$$relativeSTD = \frac{STD}{MedianDiffMinPeak} ,$$

where STD comprises the standard deviation of the emission data, MedianDiffMinPeak comprises the median of differences across the multiple sets of data, and each of the differences comprises the difference between a respective detected maximum value and a respective detected minimum value for each respective set of data of the multiple sets of data. In some embodiments, the computer-readable medium can be readable to further execute a method of determining whether to exclude one or more of the multiple sets of data based on the normalized measure; excluding sets of data from the multiple sets of data, having relativeSTD values that exceed a predetermined value, to generate processed emission data; and generating a detection signal based on the processed emission data. In some embodiments, the emission data received according to the method can comprise emission data detected from labeled nucleic acid samples, for example, during a polymerase chain reaction. In some embodiments, the method that can be executed can receive emission data from a reaction that occurs within a plurality of sample wells. In some embodiments, the emission data that is received according to the method can comprise data detected using a plurality of filters, and generating a relativeSTD can comprise generating a relativeSTD on a per-filter basis. In some embodiments, the method that can be executed can receive reaction emission data comprising multiple sets of data by generating multiple sets of data from emission data received.

### FIGURES

FIG. 1 illustrates an exemplary PCR detection system, according to various embodiments of the present teachings.

FIG. 2 illustrates an exemplary PCR amplification curve, according to various embodiments of the present teachings.

FIG. 3 illustrates well-to-well outlier or deviation analysis, according to various embodiments of the present teachings.

FIG. 4 illustrates plate-to-plate outlier or deviation analysis, according to various embodiments of the present teachings.

FIG. 5 illustrates multiple-filter readings on a sample plate, according to various embodiments of the present teachings.

FIG. 6 illustrates compensation for variations in background readings due to LED current settings in a detection system, according to various embodiments of the present teachings.

FIG. 7 illustrates compensation for variations in background readings due to variable gain settings in a detection system, according to various embodiments of the present teachings.

FIG. 8 illustrates an outlier well check, according to various embodiments of the present teachings.

FIG. 9 illustrates a uniformity check, according to various embodiments of the present teachings.

FIG. 10 illustrates overall background calibration processing, according to various embodiments of the present teachings.

### DESCRIPTION

According to various embodiments of the present teachings, methods for background calibration and outlier detection can comprise steps for identifying an outlier cycle or outlier cycles for each filter and/or each well of a sample plate used in an amplification or other reaction. Systems for carrying out calibration analysis are also provided. According to various embodiments, the calibration systems and methods can be implemented in or applied to PCR scanning systems, in which a read head containing a photodetector, for example, a photodiode or other detector, can read the fluorescent output or other output from a single well or other location, then travel to a next well or location to read the spectral dye or other output at that location, and step or repeat across a plate or other container or platform to take spectra from the entire group of sample wells, one at a time. According to various embodiments, the calibration systems and methods can be implemented in or applied to PCR imaging systems in which a photodetector, for example, a CCD, CID or other detector, images an entire plate, and all sample wells contained in that plate, at one time or at substantially one time, for instance taking a spectral image of all 96 wells of a standard microtiter plate. According to various embodiments, each well or other container or location in a plate or other platform can contain samples, for example, samples of DNA fragments or other material, to which one or more spectrally distinct dye is attached for detection and analysis.

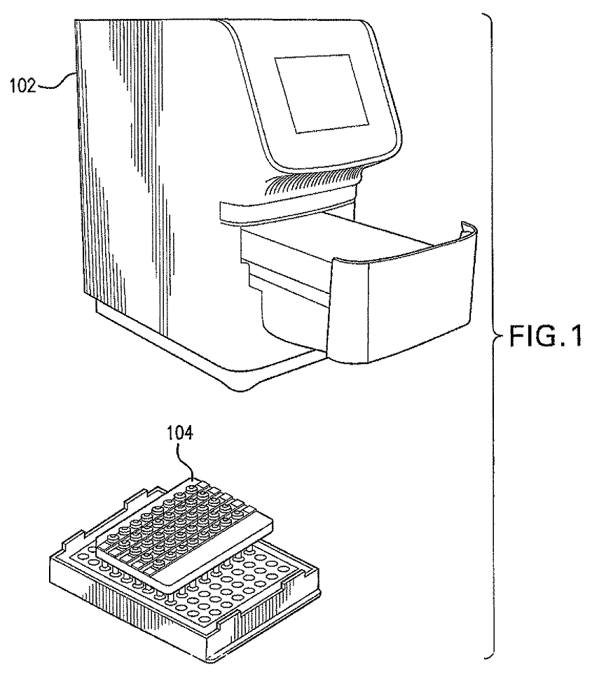

According to various embodiments, the calibration can comprise identifying a background or baseline signal detected before or during the initial stages of a PCR run, performed using a PCR system 102 reading samples contained in a plate 104, such as illustrated in FIG. 1. According to various embodiments, sample wells of plate 104 can be loaded with materials, comprising, for example, liquid running buffer, including, for instance, salts used for pH adjustment. The materials, in some embodiments, do not include sample material or fluorescent dye used to perform the background calibration analysis. The background calibration analysis can therefore capture non-reactant contributions, as well as contributions from plate 104 or surrounding material. According to various embodiments, sample wells of plate 104 can be left dry, so that only the residual or background fluorescent contribution of the plastic or other material of sample plate 104, or that of any other holder, cartridge, heater block, support, material, or structure will be captured during calibration processing. According to various embodiments, the background or baseline signal can reflect the fluorescent emission of non-amplified products, fluorescence emitted by the sample plate or by the optical elements themselves, or other anomalies or sources contributing to the noise floor or background level of the baseline signal. In some embodiments, the illumination source of PCR system 102 can be or comprise an LED, an LED array, an OLED, a floodlight source, a laser source such as a solid-state or gas-based laser, or another illumination source. According to various embodiments, the detection system of PCR system 102 can be or comprise a photodiode, a photomultiplier tube, a CCD device, or another detection device or technology. Herein, the term “emission” is used to exemplify a signal detected and/or calibrated according to various embodiments of the present teachings. It is to be understood that by “emission” the present teachings are referring to not only electromagnetic radiation but rather are also referring to any physical or chemical signal or other data that can be read, detected, imaged, or surmised from one or more area of interest, for example, a support region such as a well of a multi-well plate. “Emission” herein is intended to encompass electromagnetic radiation, optical signals, chemiluminescent signals, fluorescent signals, radiation transmission values, and radiation absorption values.

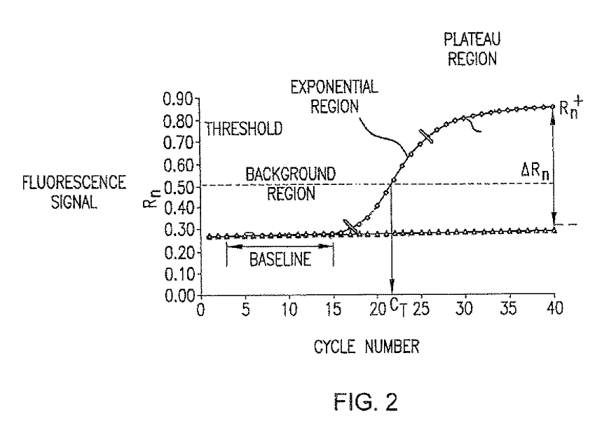

FIG. 2 illustrates an exemplary PCR amplification curve or profile resulting from a real-time PCR detection run, including an initial baseline or background region in which background, non-amplification, or non-specific signals contribute to the detected output. In general, and as, for example, illustrated in FIG. 2, a RT-PCR emission or other amplification graph, chart, or profile, will display three sections or regions: an initial baseline region, an exponential region, and a plateau region. The baseline region can display a linear, or approximately linear, or other form, over initial cycles as reaction chemistries have not liberated enough marker dye fluorescence to rise over background. The exponential region represents the rise of actual amplification product over the noise or background floor. The plateau region typically exhibits a flattening or tapering of detected emission intensities, as reactants are used up. The overall amplification profile usually shows an overall sigmoid or S-shape. Typically, RT-PCR systems determine a threshold cycle (C_{T}) representing the cycle point at which the exponential threshold is reached, and from that parameter the original sample quantity can be back-calculated using standard curves. According to various embodiments, contamination of one or more wells in plate 104 can occur, for example, as small amounts of dirt, fiber, ink, or other contaminants obstruct a well or other component, and corrupt the background reading for that well.

According to various embodiments, the calibration analysis can comprise identifying entire scanning or imaging cycles within an amplification run or runs that deviate from statistically expected ranges, and that therefore can be removed to increase the accuracy of the background characterization. According to various embodiments, accurate background characterization can contribute to increased accuracy in the readings of a PCR or other analytic run.

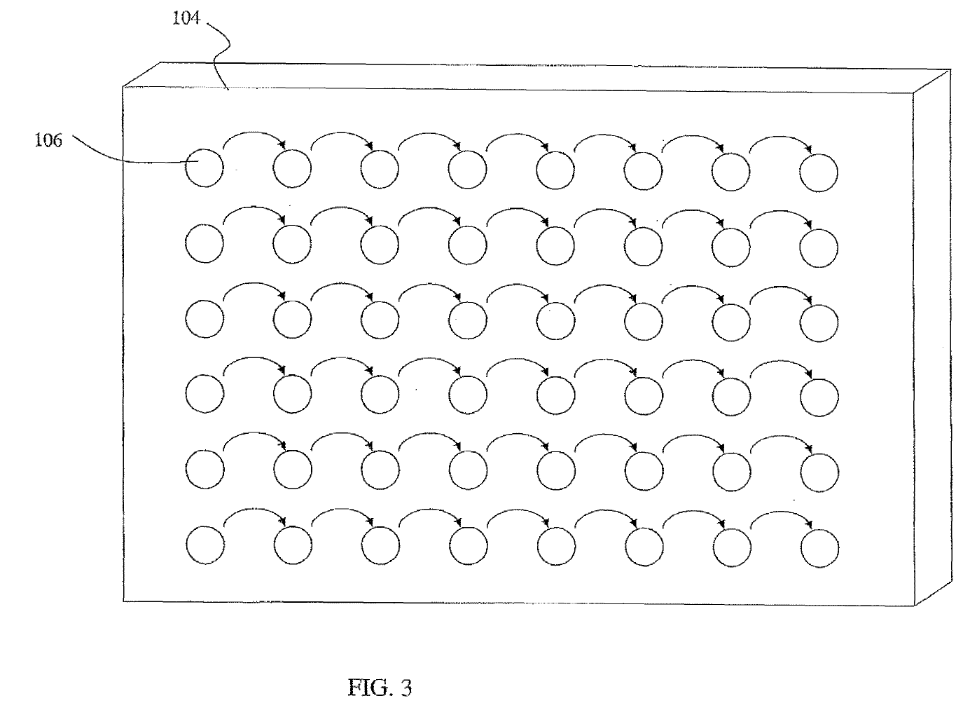

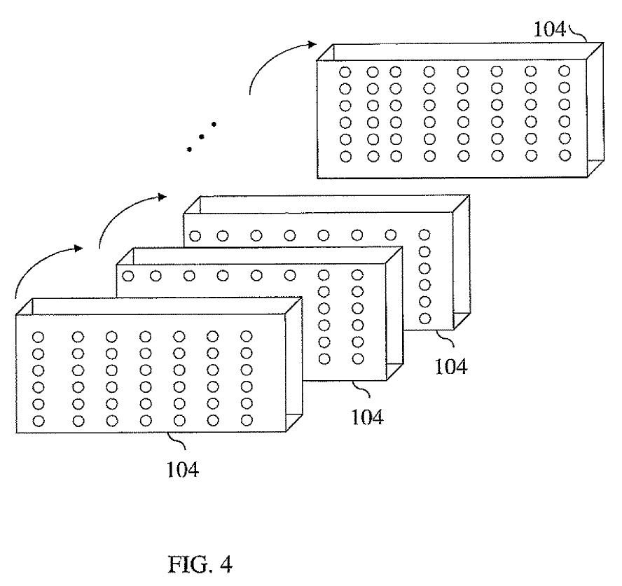

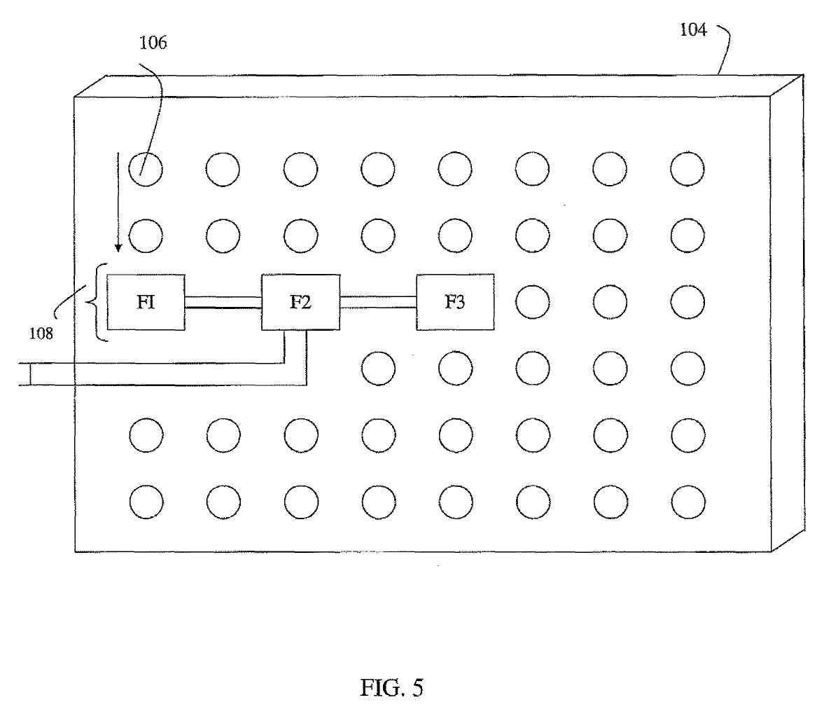

According to various embodiments, and as, for instance, illustrated in FIG. 3, background calibration can be conducted on a per-well basis to identify wells exhibiting detected emissions deviated from predetermined outlier criteria. According to various embodiments, and as, for instance, illustrated in FIG. 4, background calibration can be conducted on a per-cycle basis, in which the emission data from all wells in an entire plate is captured and processed at an aggregate level, and compared to combined data, or other data, from other plates. According to various embodiments, and as, for instance, illustrated in FIG. 5, background calibration can be conducted on a per-filter basis, in which the emission data from individual spectral filters in a set of filters 108 is captured and processed together, and compared to combined data, or other data, from other filters. According to various embodiments, any one or more of per-well, per-plate, and per-filter analyses can be combined or performed in sequence, to enhance calibration accuracy.

According to various embodiments, background calibration can comprise running a PCR or other detection cycle on some or all wells of a plate 104, or other container or support, having sample wells 106 that are loaded only with buffer. The fluorescent signal emitted from the plate itself, optical components, or other sources of residual emission, can then be detected. According to various embodiments, plate 104 can comprise 48, 96, 384 or more wells 106 arranged in a standardized, rectangular format, or those or other numbers of wells arranged in another configuration.

Systems and methods according to embodiments of the present teachings can identify an outlier cycle from a set of PCR cycles or other detection cycles. According to various embodiments, an outlier cycle can be detected using a thresholding operation, for example, determining if the captured or detected background signal is a predetermined percentage lower or higher than a computed mean signal. In various embodiments, the cycle can be labeled an outlier if the signal is at least two standard deviations away from the mean signal. According to various embodiments, if the cycle can be identified as an outlier for all filters and all wells, this cycle can be removed from further quality checking or other processing.

According to various embodiments, the background calibration analysis can comprise determining a measure of fluorescent or other baseline signal that is not sensitive to individual well, instrument, or other, variations, which variations would have an effect on metrics that are not made relative or otherwise normalized. According to various embodiments, for example, the detection system of a PCR instrument or other instrument can include an analog-to-digital converter (ADC) to convert analog optical intensity signals to digital quantities. According to various embodiments, the detection system can incorporate an amplification circuit, for example, comprising an operational amplifier (opamp) or other circuit or component, inserted before the ADC, to regulate signal levels. According to various embodiments, the opamp or other amplification circuit can be supplied with an offset or bias, to maintain a positive signal after A/D conversion is complete. According to various embodiments, the offset or bias setting of such a detection system can be set by a resistor connected to the opatnp's feedback or other circuit. As an electronic component, however, resistors can suffer from a significant degree of variability in their resistance (ohm value) rating, and depending on resistor type and cost, achieve accuracy or consistency only on the order of 5-20%, or more or less. Two machines of identical type, even if made by the same manufacturer, can therefore exhibit a variance in resistor-based offset of roughly a factor of 2, or more or less. Conducting accurate machine-to-machine PCR calibrations or other calibrations or tests can therefore be complicated or affected by electronic tolerances, such as detector offset, or other gain, sensitivity, bias, or offset settings that can not be readily determined.

According to various embodiments, for further example, the digital output of detected intensity can be processed to consist of the pixel count for a subject well or other feature, plus a predetermined or other conversion offset to be added to the raw pixel count produced by the ADC. According to various embodiments, depending on factors including initial settings, reaction chemistries and concentrations, detector and filter efficiencies, and other factors, the digital conversion offset can, in some cases, be larger than the background fluorescence or other background signal being captured and calibrated. According to various embodiments, the PCR instrument or other instrument can provide an overall sensitivity or gain setting, for example, to more readily detect comparatively faint samples or other objects. According to various embodiments, comparison of outputs from two different PCR or other instruments set to two different overall sensitivity or gain settings can therefore be difficult. According to various embodiments, the amount of current delivered to, or drawn by, the LED or other illumination source can be set at the factory or otherwise at different levels, for instance, at 200 mA, at 450 mA, or at other levels, each of which can cause the LED or other illumination source to produce a different absolute brightness. According to various embodiments, the various filters used in the detection system of a PCR instrument or other instrument can have different efficiencies across different filter wavelengths in the same instrument, or at the same wavelength across different instruments due to filter manufacturing tolerances. According to various embodiments, therefore, the absolute detected quantities taken at different wavelengths can vary in the same PCR instrument or other instrument, and conversely, emission data for the same filter wavelength captured on two of the same type of machine can vary, due to variations in filter tolerances and efficiency. These and other effects can create and introduce instrumental variance, offsets, or other kinds of unwanted bias when attempting to calibrate the background contribution of plates, wells, non-reactant liquids, filters, or other components or aspects of a PCR detection system or other detection system.

Attempting to characterize the background contributions by means of an un-normalized standard deviation or other statistical measure can therefore result in background calibration at significantly skewed scales, reducing the usability of calibration data generated in that fashion. According to various embodiments of the present teachings, instead of a conventional standard deviation calculation, a normalized or relative STD measure can be generated, permitting broader and more useful integration of well, plate, cycle, filter, machine, and other calibration measures, on a consistent basis.

According to various embodiments, the background calibration can comprise the computation of a relative standard deviation (again, referred to as “relativeSTD”) that is independent of instrument factors or effects such as instrument gain, filter efficiency, current draw (or brightness) of an LED or other illumination source, detector sensitivity, or other factors. According to various embodiments, the relativeSTD value can be defined as the ratio between the standard deviation (STD) and the measured MedianDiffMin Peak, as defined below in Equation 1.

$$relativeSTD = \frac{STD}{MedianDiffMinPeak} , \qquad Equation 1$$

where the MedianDiffMinPeak is the median of differences across all wells, where the difference is defined as the difference between maximum pixel values and the minimum pixel values of a detected emission from a well, as expressed below in Equation 2.

MedianDiffMinPeak=median(S), Equation 2

where S={max(Wᵢ)−min(Wᵢ)|I−1, . . . , 48}, and Wᵢ is a set of pixel values for pixels configured to detect fluorescence from well i.

According to various embodiments, the STD can, for example, be the standard deviation of well data from individual wells 106 of plate 104 (see FIG. 3). According to various embodiments, the STD can be, for example, the standard deviation of peak pixels or peak intensity detected for the set of individual wells 106 of plate 104.

According to various embodiments, the calibration can comprise, for each filter in a set of filters 108, or for another detection channel, identifying one or more outlier well or wells where the well background signal is lower than a threshold, as expressed below in Equation 3.

Background detected in Well(i)<mean−(2×relativeSTD)(MedianDiffMeanPeak). Equation 3

According to various embodiments, the calibration can also or instead comprise, for each filter in a set of filters 108, or for another detection channel, identifying one or more outlier well or wells where the well background signal is greater than a threshold, as expressed below in Equation 4.

Background detected in Well(i)>mean+(2×relativeSTD)(MedianDiffMeanPeak), Equation 4

where the relativeSTD is a built-in or defined parameter, for instance, generated according to Equation 1 above, and the MedianDiffMeanPeak is generated according to Equation 2 above, and, for instance, is calculated from the background calibration run in a PCR or other amplification or other reaction.

According to various embodiments, the background calibration can comprise, for each filter in a set of detection filters 108 and/or for each well 106, calculating the background well signal by averaging the background signal for cycles that are not identified or excluded as outliers and are instead considered useful or reliable.

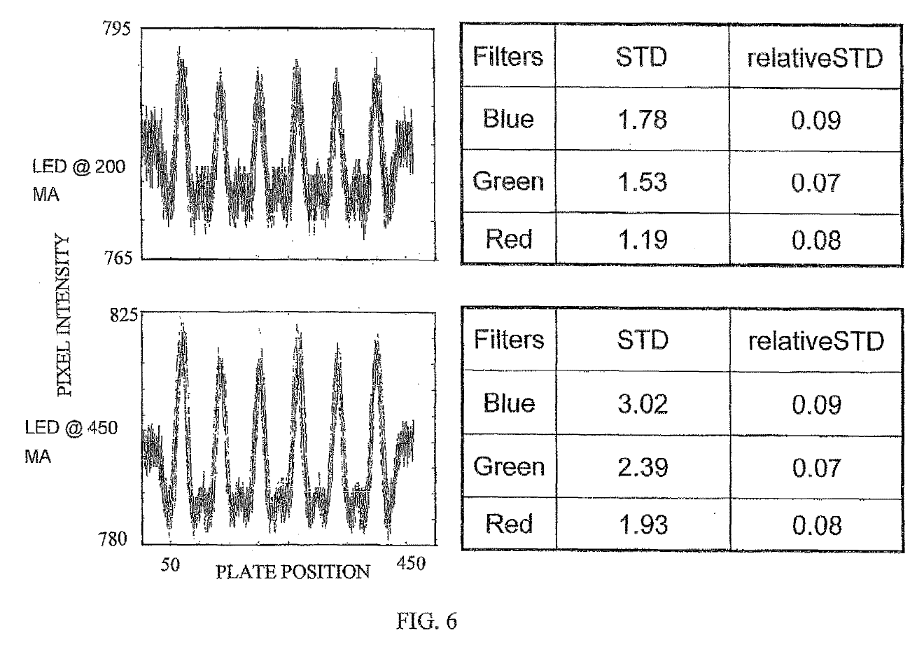

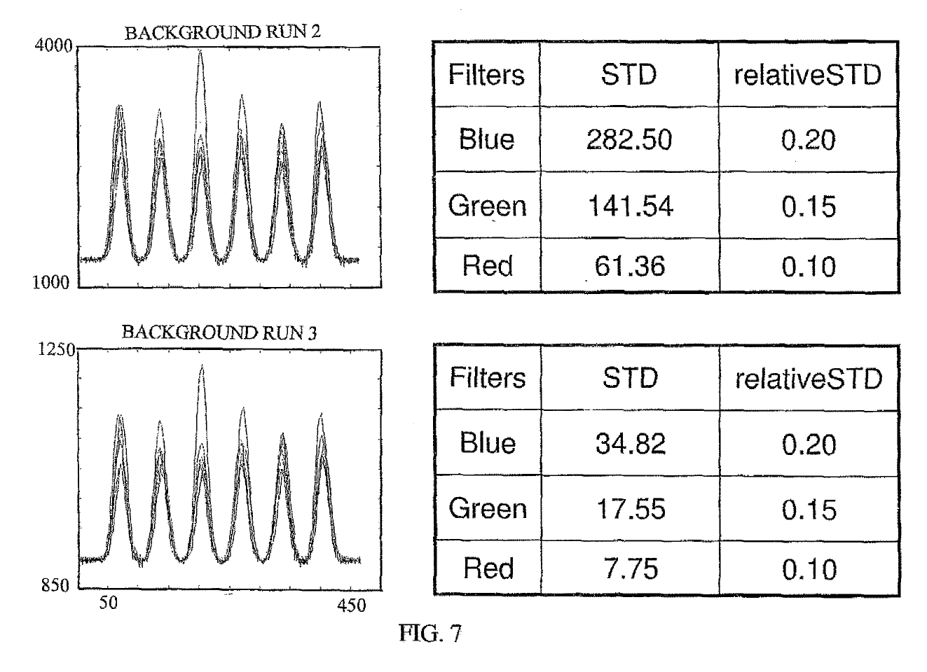

According to various embodiments of the present teachings, the defined measure of MedianDiffMeanPeak and associated relativeSTD can cancel or compensate for the effect of instrumental variations that can come into play across different instruments, plates, or processes. As, for example, illustrated in FIG. 6, according to various embodiments, those instrumental variations can include, for example, differing settings on the draw current and therefore illumination brightness of the LED or other illumination source. While measurements taken in conventional standard deviation calculations as shown differ in absolute amount, the relativeSTD measure on background signal variation is normalized (for each of three color filters), and in this case, as shown, is equalized across different LED current settings. As, for example, illustrated in FIG. 7, according to various embodiments, those instrumental variations can include, for example, differing settings on gain of the detector and therefore different detected absolute intensity values. While intensity or amplitude measurements taken in conventional standard deviation calculations differ in absolute amount, the relativeSTD measure on background signal variation is normalized (for each of three color filters), and in the case shown, are equalized across different detector gain, amplification, or sensitivity settings.

According to various embodiments, employing a conventional standard deviation computation can ultimately produce inconsistent results, since different instruments can be set to different amplification or gain values, can use LEDs or other illumination sources which are set to be driven with different amounts of current and/or have different efficiencies and thus produce different brightness, can have photodetectors of different sensitivities or efficiencies, or can otherwise differ in factors that affect the absolute value or amplitude of the output. According to various embodiments, the background calibration techniques described herein instead rely upon metrics for measuring background variation that are invariant under various instrumental fluctuations or deviations, and therefore permit comparison between instruments, plates, cycles, or other items that may or may not operate with the same instrumental biases.

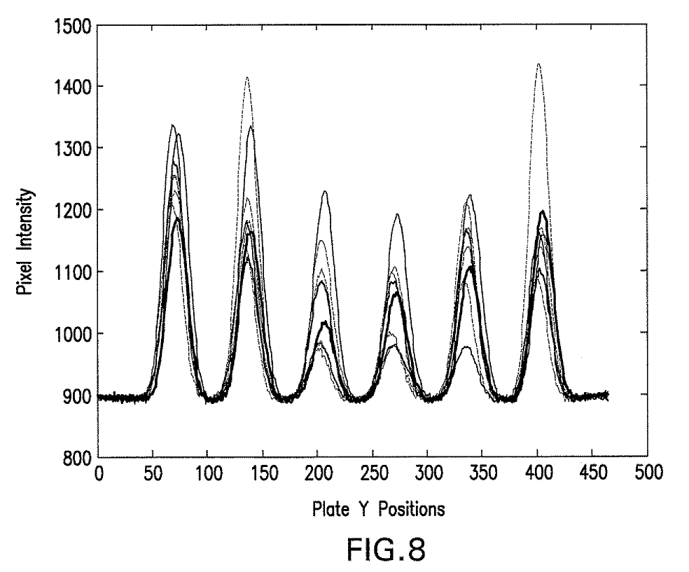

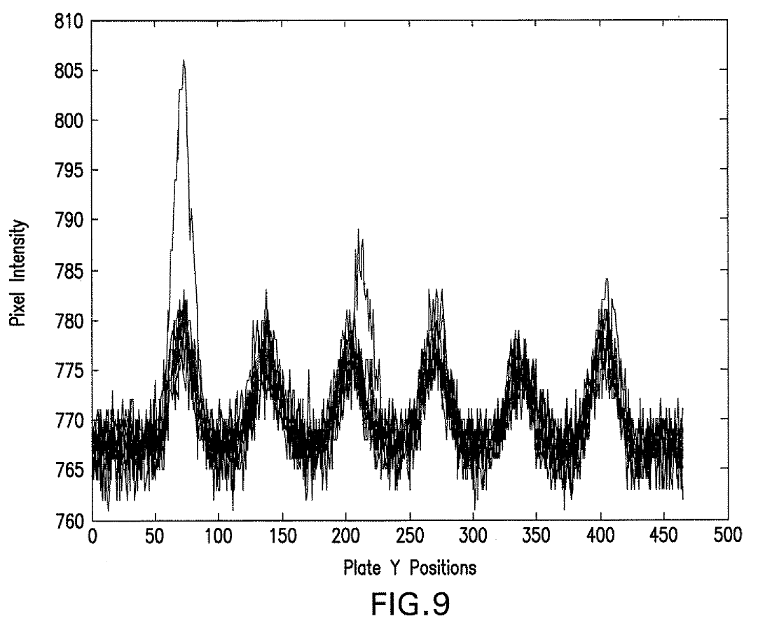

According to various embodiments, the background calibration can utilize metrics for measuring background variation that do not depend on absolute values of detected background output, but instead, for example, can utilize relative values, such as a ratio or other scaled or transformed value or values. The relativeSTD described above meets all requirements for invariance. As, for example, illustrated in FIG. 8, according to various embodiments, the relativeSTD measure or metric and can be used to detect individual outlier wells consistently across multiple plates, PCR instruments, or other hardware units, because a common or normalized measure of pixel intensity readings is produced. As, for example, illustrated in FIG. 9, according to various embodiments, the relativeSTD measure or metric can also be used to check background or baseline signal uniformity within defined positions of one plate, or across multiple plates.

According to various embodiments, the relativeSTID can be taken, for instance, of either well intensity data as a whole, or peak pixel density for a single pixel with greatest amplitude corresponding to a given well. According to various embodiments, the detector system within a PCR system 102 or other instrument can have imaging resolution of hundreds of lines per inch, such as 400 lines per inch, or more or less, so that a significant number of separate intensities based on narrow line widths can be reported moving across an individual well 106. According to various embodiments, use of the standard deviation of peak pixels improves on the calculation of standard deviation based on overall or total well signal, because the variance of standard deviation of total well data will therefore typically be several times higher than the variance of standard deviation of single peak pixels, due to increased sampling for the total well data.

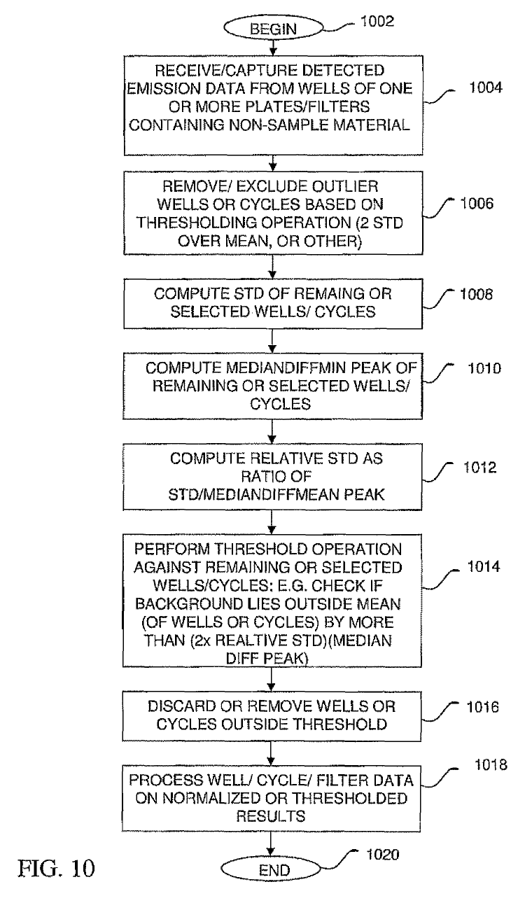

Overall fluorescent background calibration processing according to various embodiments of the present teachings is illustrated in the flowchart of FIG. 10. Processing can begin in step 1002. In step 1004, the detected fluorescent or other emission data from dry or wet sample wells lacking sample material can be captured, received, or detected, for example, from a PCR instrument or other instrument or system. In step 1006, outlier wells or cycles can be excluded or removed, for example based on predetermined, user-selected, dynamically calculated, or other thresholding operations. In step 1008, the standard deviation of the remaining or selected wells or cycles can be computed.

In step 1010, the MedianDiffMinPeak quantity can be computed or generated, for example, according to Equation 2 above. In step 1012, the relativeSTD quantity or variable can be computed or generated, for example, according to Equation 1 above. In step 1014, a thresholding operation can be performed on the remaining or selected wells or cycles, for instance, according to Equations 3 and 4 above. In some embodiments, other thresholding equations or quantities can be used. In step 1016, wells or cycles lying outside the thresholding limits of step 1014 can be removed or discarded. In step 1018, the normalized or conditioned well, cycle, filter, or other data, can be used to process analytical PCR runs, or perform other operations. In step 1020, processing can end, repeat, return to a prior processing point, or proceed to a further processing point.

According to various embodiments, the background calibration can comprise reporting a problem with the background run such as detected non-uniform results, and/or outlier wells. In some embodiments, the problem can be reported to an operator, to an automated logging system, or to another destination, location, or storage. According to various embodiments, the quality check reflected in background or baseline calibration that identifies outliers is consistent and robust across different instruments, and is insensitive to instrument factors such as instrument gain, LED current, filter design, and offset. According to various embodiments, the background calibration can produce improved results by averaging cycle data classified as accurate or reliable, and removing outlier cycles.

According to various embodiments, different aspects of the differential dissociation/melting curve analysis of the present teachings can be applied to commercial systems and implementations, for example, can be applied to the STEPONE™ system commercially available from Applied Biosystems, Foster City, Calif., and described, for example, in the publication entitled “Applied Biosystems Step One Real-Time PCR System Getting Started Guide,” which publication is incorporated by reference in its entirety herein.

It will be appreciated that while various embodiments described above involve the calibration of one or more aspects of background or baseline signal behavior, according to various embodiments, more than one type of background or other calibration can be performed, together or in sequence.

Various embodiments of the present teachings can be implemented, in whole or in part, in digital electronic circuitry, or in computer hardware, firmware, software, or in combinations thereof. Apparatus of the present teachings can be implemented in a computer program, software, code, or algorithm embodied in machine-readable media, such as electronic memory, CD-ROM or DVD discs, hard drives, or other storage devices or media, for execution by a programmable processor. Various method steps according to the present teachings can be performed by a programmable processor executing a program of instructions to perform functions and processes according to the present teachings, by operating on input data and generating output. The present teachings can, for example, be implemented in one or more computer programs that are executable on a programmable system including at least one programmable processor coupled to receive data and instructions from, and to transmit data and instructions to, a data storage system or memory, at least one input device such as a keyboard and mouse, and at least one output device, such as, for example, a display or printer. Each computer program, algorithm, software, or code, can be implemented in a high-level procedural or object-oriented programming language, or can be implemented in assembly, machine, or other low-level language, if desired. According to various embodiments, the code or language can be a compiled, interpreted, or otherwise processed for execution.

Various processes, methods, techniques, and algorithms disclosed herein can be executed on processors that can include, by way of example, both general and special purpose microprocessors, for example, general-purpose microprocessors such as those manufactured by Intel Corp. or AMD Inc., digital signal processors, programmable controllers, or other processors or devices. According to various embodiments in general, a processor will receive instructions and data from a read-only memory and/or a random access memory. In some embodiments, a computer implementing one or more aspects of the present teachings can generally include one or more mass storage devices for storing data files, such as magnetic disks, such as, internal hard disks, removable disks, magneto-optical disks, and CD-ROM, DVD, Blu-Ray, or other optical disks or media. Memory or storage devices suitable for storing, encoding, or embodying computer program instructions or software and data as described herein can include, for instance, all forms of volatile and non-volatile memory, including, for example, semiconductor memory devices, such as random access memory, electronically programmable memory (EPROM), electronically erasable programmable memory (EEPROM), and flash memory devices, as well as magnetic disks such as internal hard disks and removable disks, magneto-optical disks, and optical disks. Any of the foregoing can be supplemented by, or incorporated in, ASICs. According to various embodiments, processors, workstations, personal computers, storage arrays, servers, and other computer, information, or communication resources, used to implement features of the present teachings, can be networked or network-accessible.

Other embodiments will be apparent to those skilled in the art from consideration of the present specification and practice of the present teachings disclosed herein. For example, resources described in various embodiments as singular can, in embodiments, be implemented as multiple or distributed, and resources described in various embodiments as distributed can be combined. It is intended that the present specification and examples be considered as exemplary only.

What is claimed is:

1. A method of generating a detection signal from a reaction, comprising:
receiving reaction emission data comprising multiple sets of data;
generating a relative standard deviation (relativeSTD) representing a normalized measure of variation in the multiple sets of data, by applying the equation:
$$relativeSTD = \frac{STD}{MedianDiffMinPeak} ,$$
where STD comprises the standard deviation of the emission data, MedianDiffMinPeak comprises the median of differences across the multiple sets of data, and each of the differences comprises the difference between a respective detected maximum value and a respective detected minimum value for each respective set of data of the multiple sets of data;
determining whether to exclude one or more of the multiple sets of data based on the normalized measure;
excluding sets of data from the multiple sets of data, having relativeSTD values that exceed a predetermined value, to generate processed emission data; and
generating a detection signal based on the processed emission data.

2. The method of claim 1, wherein the emission data comprises emission data detected from a biological sample.

3. The method of claim 1, wherein the reaction comprises a polymerase chain reaction.

4. The method of claim 1, wherein reaction comprises a reaction with a nucleic acid.

5. The method of claim 1, wherein the receiving emission data comprises receiving emission data from a plurality of sample wells.

6. The method of claim 1, wherein the emission data comprises data detected using a plurality of filters, and the generating a relativeSTD comprises generating a relativeSTD on a per-filter basis.

7. A system for generating a detection signal from a reaction, comprising:
an input unit configured to receive reaction emission data comprising multiple sets of data; and
a processor unit, the processor unit being configured to
generate a relative standard deviation (relativeSTD) representing a normalized measure of variation in the multiple sets of data, by applying the equation:
$$relativeSTD = \frac{STD}{MedianDiffMinPeak} ,$$
where STD comprises the standard deviation of the emission data, MedianDiffMinPeak comprises the median of differences across the multiple sets of data, and each of the differences comprises the difference between a respective detected maximum value and a respective detected minimum value for each respective set of data of the multiple sets of data,
determine whether to exclude one or more of the multiple sets of the data based on the normalized measure,
exclude sets of data from the multiple sets of data, having relativeSTD values that exceed a predetermined value, to generate processed emission data, and
generate a detection signal based on the processed emission data.

8. The system of claim 7, wherein the emission data comprises emission data detected from labeled nucleic acid samples.

9. The system of claim 7, wherein the reaction comprises a polymerase chain reaction.

10. The system of claim 7, wherein the input unit comprises a plurality of different filters configured to filter the emission data from the reaction.

11. A background calibration profile, the background calibration profile being generated by a method comprising:
receiving reaction emission data comprising multiple sets of data;
generating a relative standard deviation (relativeSTD) representing a normalized measure of variation in the multiple sets of data, by applying the equation:
$$relativeSTD = \frac{STD}{MedianDiffMinPeak} ,$$
where STD comprises the standard deviation of the emission data, MedianDiffMinPeak comprises the median of differences across the multiple sets of data, and each of the differences comprises the difference between a respective detected maximum value and a respective detected minimum value for each respective set of data of the multiple sets of data;
determining whether to exclude one or more of the multiple sets of data based on the normalized measure;
excluding sets of data from the multiple sets of data, having relativeSTD values that exceed a predetermined value, to generate processed emission data; and
generating a background calibration profile based on the processed emission data.

12. The background calibration profile of claim 11, wherein the emission data comprises emission data detected from labeled nucleic acid samples.

13. The background calibration profile of claim 11, wherein the emission data comprises emission data detected from fluorescently labeled DNA samples.

14. The background calibration profile of claim 11, wherein the amplification reaction comprises a polymerase chain reaction.

15. The background calibration profile of claim 11, wherein the receiving emission data comprises receiving emission data from DNA samples loaded in a plurality of sample wells.

16. A computer-readable medium, the computer-readable medium being readable to execute a method of generating a detection signal from a reaction, the method comprising:
receiving reaction emission data comprising multiple sets of data;
generating a relative standard deviation (relativeSTD) representing a normalized measure of variation in the multiple sets of data, by applying the equation:
$$relativeSTD = \frac{STD}{MedianDiffMinPeak} ,$$
where STD comprises the standard deviation of the emission data, MedianDiffMinPeak comprises the median of differences across the multiple sets of data, and each of the differences comprises the difference between a respective detected maximum value and a respective detected minimum value for each respective set of data of the multiple sets of data;
determining whether to exclude one or more of the multiple sets of data based on the normalized measure;
excluding sets of data from the multiple sets of data, having relativeSTD values that exceed a predetermined value, to generate processed emission data; and
generating a detection signal based on the processed emission data.

17. The computer-readable medium of claim 16, wherein the emission data comprises emission data detected from labeled nucleic acid samples.

18. The computer-readable medium of claim 16, wherein the reaction comprises a polymerase chain reaction.

19. The computer-readable medium of claim 16, wherein the reaction occurs within a plurality of sample wells.

20. The computer-readable medium of claim 16, wherein the emission data comprises data detected using a plurality of filters, and the generating a relativeSTD comprises generating a relativeSTD on a per-filter basis.
